(19) United States
(12) Patent Application Publication
(10) Pub. No.: US 2005/0118241 A1
(43) Pub. Date: Jun. 2, 2005

(54) Platform for transdermal formulations (ptf)

(76) Inventor: Yuval Simha Landschaft, Munich (DE)

Correspondence Address: D Peter Hochberg Company;Baker Building, 1940 East 6th St., 6th Floor, Cleveland, OH, 44114-2294, US

(21) Appl. No.: 10/511,463

(22) PCT Filed: Jun. 21, 2003
(86) PCT No.: PCT/IB03/03467

(30) Foreign Application Priority Data
Jun. 27, 2002 (DE) 102286809

(51) Int. Cl. A61K 38/17; A61L 15/16

(52) U.S. Cl. 424/448; 514/2

(57) ABSTRACT

A composition which can be used as platform for transdermal administration of therapeutically active compounds and/or nutrients. The composition comprises at least one therapeutically active compound and/or at least one nutrient, and a non-oily emulsion.

### CROSS REFERENCE TO RELATED APPLICATIONS

This application is a National Stage application of International Application No. PCT/IB2003/03467, filed on Jun. 21, 2003, which claims priority of German Application No. 102 28 680.9, filed Jun. 27, 2002.

### DESCRIPTION OF THE PRIOR ART

This invention relates to compositions that allow permeation of small molecules, ionic compounds and polypeptides through the skin and their use for the manufacture of a medicament that can be utilized to treat specific conditions and diseases in humans and animals by transdermal delivery of medicinals, therapeutically active agents and/or nutritional agents.

As it is well known, transdermal delivery of pharmaceutically active ingredients which are absorbed through the skin into the underlying blood vessels has the advantage over conventional administration forms for oral or other parenteral applications of providing a controllable plasma level in the therapeutic range while at the same time avoiding the therapeutic dose to fall short or be exceeded. Hence, transdermal drug delivery is a convenient and reliable form of administering medicinals.

Transdermal delivery may particularly benefit patients with chronic disease. Many such patients have difficulty following regimens requiring several doses daily of medications that repeatedly cause unpleasant symptoms. They find the same drugs much more acceptable when administered in transdermal systems that require application infrequently—in some cases, only once or twice weekly—and that reduce adverse effects.

Transdermal delivery has been referred to in many circles as the “delivery system of the future”. In the last few years, medical researchers have realized that many nutrients are more effectively delivered via the skin (the body largest organ) than by oral means. Many nutrients cannot be effectively absorbed when taken orally because the stomach acids destroy them and/or the liver discards them. Transdermal delivery absorbs more than 90% of most hormones compared to less than 5% when taken orally.

A further advantage of transdermal drug delivery lies in the fact that the gastro-intestinal tract and the portal system are circumvented. As a result, it is not necessary to take into account the first-pass effect, which requires high doses of medicinal agents within oral administration forms. Such high doses of medicinal agents are often responsible for plasma peaks involving undesired side effects.

Transdermal application permits the use of a much broader range of drugs and natural substances for therapeutic application, in particular drugs which have short half-lives in the body such as hormones. Such substances would have to be taken many times daily by other ordinary dosage forms. Continuous transdermal delivery provides a practical way of administration and one that can mimic the body's own patterns of secretion.

In summary, transdermal drug delivery, as compared to “traditional” routes, has the following advantages:
It avoids the need for gastrointestinal absorption.
It avoids the first-pass effect.
It permits multiple therapies with single application.
It extends the activity of drugs with short half-lives.
In many cases, it provides the capacity to terminate drug effects rapidly.
It allows rapid identification of the medication in emergency situations.

Typical systemically active agents that may be delivered transdermally are therapeutic agents that are sufficiently potent and thus can be delivered through the skin to the blood steam in sufficient quantities to produce the desired therapeutic effect. In general, this includes therapeutic agents in all of the major therapeutic areas. The major restriction for the treatment of specific conditions and diseases by transdermal delivery of medicinal agents is the capability of medicinals to be absorbed through the skin. Many known medicinals are either not skin absorbable or are absorbed at rates insufficient for therapeutic purposes. In particular, transdermal delivery of either ionic compounds or large polypeptides has not been achieved successfully yet.

Considerable research efforts have been invested in the attempts to deliver polypeptides and ions through the skin. Most of the solutions that were suggested involved complicated and expensive methods. A recent review summarized a large part of these studies with an emphasis on a simple method that was referred to as “lipid-base delivery system”, see: Foldvari, M et al., Biotechnol. Appl. Biochem., 30:129-137 (1999).

Several transdermal therapeutic devices exist and are marketed, however, all exist as products for “small” molecular weight drugs and non-ionic compounds. Transdermal therapeutic devices for delivery of either ionic compounds or large polypeptides do not exist yet.

### SUMMARY OF THE INVENTION

It was, therefore, a main object of the present invention to provide a composition for the manufacture of medicaments which provide improved transdermal delivery of molecules and drugs, in particular of polypeptides and/or ionic compounds.

It has surprisingly been found that a non-oily emulsion can provide rapid permeation of an active ingredient through the skin and into the blood vessels, wherein the active ingredient can be selected, for example, from the group comprising small molecules, ionic compounds and polypeptide hormones.

An advantage of the non-oily emulsion is that ionic compounds (e.g. ferrous ions) and polypeptides with a molecular weight of up to 7000 Dalton such as, for example, insulin are enabled to permeate the skin. A further advantage of the non-oily emulsion is the remarkably quick absorption of an active ingredient into the circulation.

In a preferred embodiment of the composition, the non-oily emulsion comprises a mixture of lecithin(s), bile salts and cholesterol in water.

Lecithins are glycero-phospholipids that are formed by fatty acids, glycerol, phosphoric acid and choline. Naturally occurring lecithins are derivatives of the 1,2-diacyl-sn-glycerol-3-phosphoric acids. The large number of different lecithins results from varying fatty acid residues. One always obtains a mixture of lecithins when they are extracted from biological material.

Bile salts are the salts of substituted cholanic acids, which are associated primarily with glycine or taurins in bile. Cholanic acid itself is not present in bile.

Cholesterol is the major representative of the zoosterols and can be found in virtually all organisms.

Each of the components of the non-oily emulsion, lecithin(s), bile salts and cholesterol, is preferably present in an amount of between 2 to 15% (w/v), in relation to the non-oily emulsion. It is particularly preferred that the components of the mixture are present in a ratio by weight of 2:1:1 (lecithin:bile salt:cholesterol).

It is preferred that the sum of the amounts of lecithins, bile salts and cholesterol constitutes 6-30% (w/v) of the non-oily emulsion.

In a preferred embodiment, the composition for transdermal administration of therapeutically active compounds and/or nutrients further comprises an organic sulfur compound.

The organic sulfur compound is preferably present in an amount of 2-30% (w/v) and more preferably in an amount of % (w/v), in relation to the non-oily emulsion.

The organic sulfur compound is preferably selected from the group comprising dimethylsulfoxide, methylsulfonylmethane (MSM), 2,3-dimethylsulfolane and 2,4-dimethylsulfolane and sodium lauryl sulfate, wherein MSM is particularly preferred.

U.S. Pat. No. 6,183,758 discloses a skin absorbent cream comprising of a combination of two separate solutions. The first solution consists of water, MSM and urea. The other solution contains propylene glycol and a medication or molecular organic compound such as a steroid, alkaloid or nutrient.

The composition for transdermal administration of active compounds according to the present invention has universal applications and can serve as platform for the manufacture of medicaments for transdermal delivery of molecules and drugs such as non-steroidal anti-inflammatory drugs (NSAIDs), e.g. ibuprofen. The composition is particularly suitable in assisting the permeation of polypeptides with a molecular weight of up to 7,000 Dalton and/or ionic compounds.

Examples for polypeptides that can be administered through the skin utilizing the composition of the present invention are insulin, glucagon, calcitonin and various other peptide hormones.

For a review on the transport of peptide and protein drugs through the intestinal, buccal, nasal and pulmonary absorptive membranes, as well as transdermal penetration, see: Verhoef, J. C., Eur. J Drug Metab. Pharmacokinet., 15(2): 83-93 (1990).

Examples for ionic compounds that can be administered through the skin utilizing the composition of the present invention are ferrous fumarate, ferrous sulphate, ferrous glutamate, calcium, zinc and various other ions.

Insulin, in particular, is extremely important for the treatment of Diabetes Mellitus, a serious pathologic condition that represents the 4^{th} leading cause of death in the United States. Diabetes Mellitus, due to inadequate insulin secretion or lack of insulin is extremely widespread. Treatment of this disease (especially under severe conditions) by injections or infusion of insulin is predominantly via the subcutaneous route and to a minor extent via the intravenous or intramuscular routes. These methods of administration have the disadvantage that once given they cannot be withdrawn, e.g. in cases of hypoglycemia or other adverse effects. It is important to note that up to 7% of deaths in insulin-dependent diabetics have been attributed to hypoglycemia. Although subcutaneous insulin replacement therapy has saved countless lives, it has become clear over the last few years that this non-physiologic insulin administration is far from being optimal in forestalling cardiovascular and neurological complications associated with the disease.

Thus, there would be a great advantage in administering insulin (and for that matter other proteins and polypeptides) transdermally, in that a transdermal patch can be readily withdrawn in case of adverse effects developing in a patient. Moreover, transdermal administration is a more convenient and user-friendly mode of drug administration, compared to, e.g., the daily injection regimen which is widely used clinically at the present time in the case of insulin. Extensive research into the administration of insulin, either by transdermal or by other routes, has not resulted in any practical and simple clinical use till now.

Various techniques were tested and described in the literature for the delivery of insulin through the skin. For example, the ultrasonic vibration technique, tested on hairless mice immersed in a bath of neutral aqueous insulin, see: Tachibana, K. et al., J. Pharm. Pharmacol., 43(4): 270-1 (1991). The use of various emulsions of insulin in treating rabbits or diabetic rats was described by Shichiri, M et al., in Diabetologia, 10: 317-21 (1974) and in Diabetes, 24: 971 (1975). Transdermal delivery of insulin in mice by using lecithin vesicles as a carrier was recently described by Guo, J. et al., Drug Deliv., 7(2): 113-116 (2000). Many other examples, none leading so far to practical solution, were described in the open literature.

With reference to the attached Figures, it is illustrated that the PTF of the present invention is a safe and efficient way to administer medicinals through the skin of a patient. In particular, the PTF of the present invention allows ionic compounds and peptide hormones to be administered readily through the patient's skin.

### BRIEF DESCRIPTION OF THE FIGURES

FIG. 1: The effect of the novel insulin PTF on plasma glucose levels in a healthy subject.

FIG. 2: The effect of the novel insulin PTF on plasma glucose concentration (percent change from baseline levels) following 75 g sugar load, in a healthy subject, with and without insulin patch.

FIG. 3: The effect of an acute administration of the novel insulin PTF on plasma glucose levels of a type II diabetic subject.

FIG. 4: The effect of two prolonged applications of the novel insulin PTF on plasma glucose levels of a type II diabetic subject.

FIG. 5: The effect of glucagon in the novel PTF on plasma glucose levels (percent change versus time zero) following 75 g sugar load in a healthy subject.

FIG. 6: The effect of ferrous sulfate containing patch, applied to calves' ears, on ferrous concentration in their plasma.

FIG. 7: The effect of ibuprofen containing patch, applied to rabbits' skin, on their plasma ibuprofen concentration.

FIG. 8: Calcitonin plasma concentrations in calves, following application of a patch with PTF containing calcitonin (averages±SEM).

FIG. 9: The effect of calcitonin containing PTF patch on the concentration of calcium in calves.

FIG. 10: Triclabendazole (TCBZ) concentration in plasma of cows treated with PTF patch, with and without ivermectin (averages±SD; N=5).

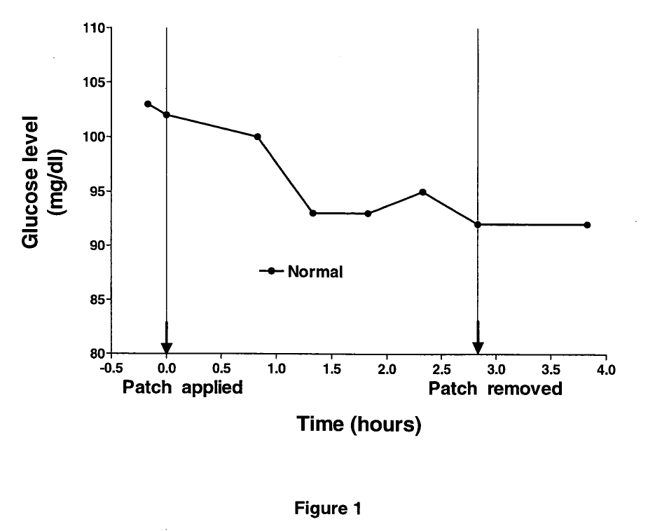

FIG. 1: Ivermectin concentration in plasma of cows treated with PTF patch, with and without triclabendazole (TCBZ) (averages±SD; N=5).

The efficiency, safety and universal applicability of the PTF of the present invention will be illustrated by way of examples. It should be understood that these examples are in no way restricting the present invention.

### DETAILED DESCRIPTION OF THE INVENTION

### EXAMPLE 1

A patch of the novel PTF was soaked with an emulsion of the present invention comprising of a non-oily emulsion, MSM and insulin (formula I). The patch was applied to a healthy volunteer after establishing the subject's glucose baseline. Glucose baseline was determined to be approx. 102 mg/dl (mg %). Subsequent blood glucose levels were measured approximately half an hour apart. FIG. 1 illustrates that blood glucose concentration was reduced by 5 to 8%.

Such a moderate decline in blood glucose concentration could be attributed to a feedback mechanism that decreases the synthesis and secretion of endogenous insulin. This result demonstrates the safety quality of transdermal application of insulin utilising the non-oily emulsion of the invention, because it is unlikely that hypoglycemia will occur upon inadvertent use of an insulin patch based on the transdermal formulation platform of the invention.

### EXAMPLE 2

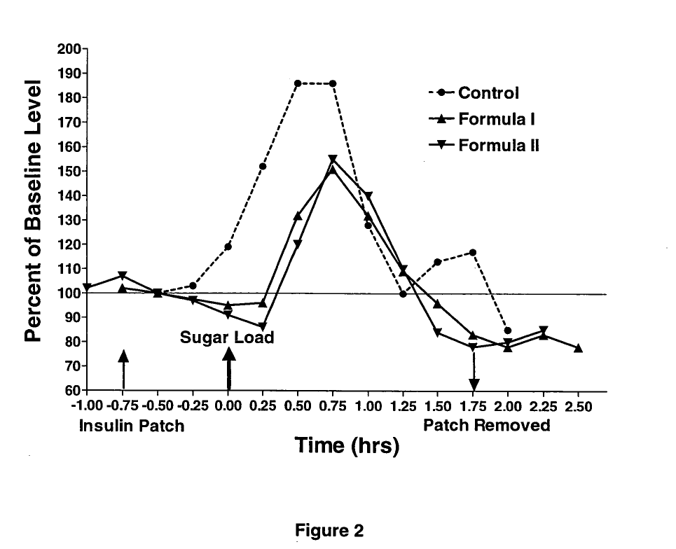

The PTF containing insulin in the specific non-oily emulsion (formula I) did not exhibit a major effect on blood glucose level when applied to a normal healthy subject (example 1). To demonstrate the efficiency of the transdermal formulation platform of the invention, it was further tested on another healthy subject that was loaded with 75 g sugar. After establishing glucose baseline of the healthy volunteer, the subject was loaded with 75 g of sugar dissolved in water. Blood glucose levels were monitored for the next two hours. In another experiment with the same subject, at least one week apart, the PTF patch soaked with the emulsion according to formula I was applied for half an hour (FIG. 2) and then the subject was offered an identical sugar load of 75 g in water.

As can be seen in FIG. 2, the area under the curve for glucose concentration over time (in which baseline levels were assigned the value of 100%) was about 50% smaller for the sugar load following application of the insulin patch compared to the area after a control sugar load.

### EXAMPLE 3

A similar experiment to example 2 was repeated in the same healthy subject, except that MSM was omitted from the non-oily emulsion (FIG. 2, Formula II). A patch, which was soaked with the non-oily emulsion made of lecithins, bile salts and cholesterol containing insulin, was applied to the subject. Almost one hour later, the subject was loaded with 75 g of sugar dissolved in water. Blood glucose levels were monitored for one and a half hours. The PTF patch was removed and at this point the subject was approximately 20% hypoglycemic compared to his own baseline. As seen in FIG. 2, the area under the curve for glucose concentration over time was similar to that for Formula I and significantly lower than the area under the curve for the control sugar load. In this specific case, the non-oily emulsion without MSM worked almost equally well as the one containing MSM.

### EXAMPLE 4

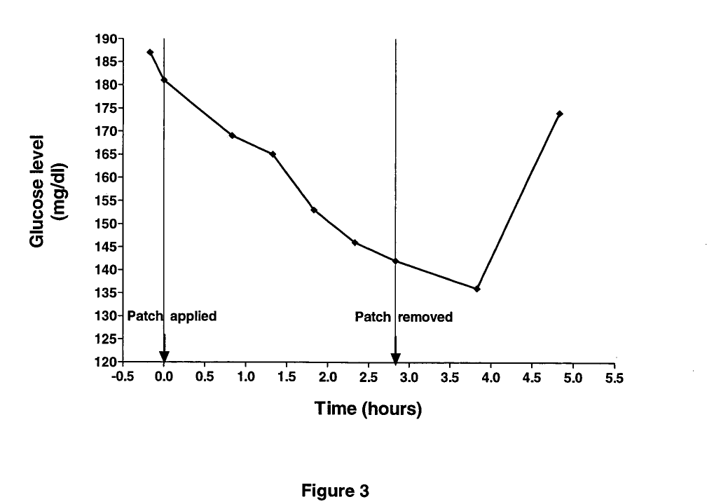

A PTF patch was soaked with an emulsion of the present invention comprising of a non-oily emulsion, MSM and insulin (formula I). The patch was applied onto a type II diabetic subject, who was regularly treated with a biguanide drug (metformin hydrochloride, 850 mg t.i.d.), and a sulfonylurea drug (repaglimide, 2 mg t.i.d.), with no insulin treatment. The morning of testing the subject did not take any drug treatment and started with baseline glucose concentration around 184 mg/dl. Following application of the patch, glucose concentration gradually decreased during the next three hours by 23% (see FIG. 3). At this point the PTF patch was removed. During the next hour an additional decrease of 3% in blood glucose was observed which might be due to an insulin depot in the skin. However an hour later and after consuming some food, the levels increased again to the starting high levels. At this stage the subject resumed his regular drug treatment.

These experimental data unequivocally demonstrate the efficiency of the PTF in transdermal administration of insulin.

### EXAMPLE 5

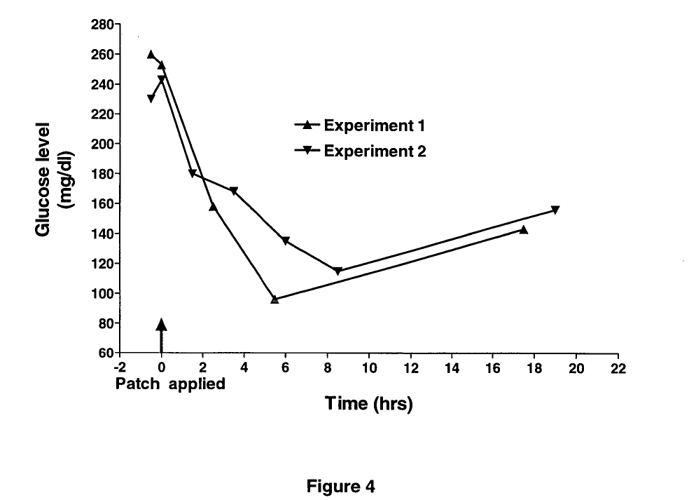

In two experiments, in two different occasions, another type II diabetic subject was treated for a whole day with prolonged application of one patch of the PTF, soaked with an emulsion of the present invention comprising of a non-oily emulsion, MSM and insulin. The subject was regularly under treatment of a sulfonylurea drug (glibenclamide, 5 mg b.i.d.) with no insulin treatment. On testing days he took no medication and started with baseline glucose levels in the range of 240-260 mg/dl. 4-8 hours following patch application his glucose levels were in the normal range (see FIG. 4). After each experiment, following one day of treatment with the patch, the subject reported glucose levels around 150 mg/dl with his usual drug therapy.

### EXAMPLE 6

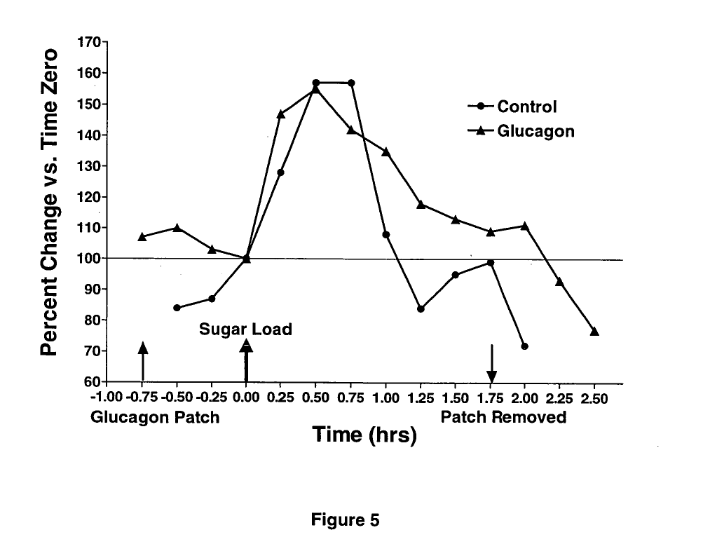

The universality of the non-oily emulsion of the invention in inducing skin penetration of peptides is demonstrated in another example. Glucagon is a 3.5 kDa peptide whose high levels are known to inhibit glycolysis and stimulate gluconeogenesis. It is extensively degraded in liver, kidney and plasma and therefore its half-life is 3-6 minutes. To demonstrate the penetration of glucagon through the skin, the following experiment was conducted on a healthy volunteer. On two different occasions, the percent change in plasma glucose compared to time zero (time of sugar load) was followed in the same volunteer after 75 g of sugar load, with and without glucagon PTF (applied 45 minutes before the sugar load). Patch application prolonged dramatically the duration of the decrease in glucose concentration, which declined rapidly only following patch removal (see FIG. 5).

### EXAMPLE 7

The non-oily emulsion in the PTF of the invention is extremely effective in enhancing the penetration of ions through the skin, as shown in the following example.

The bioavailabilty of iron and adverse effects of its oral administration are a source of continuing concern, e.g. see: Thorand, B, et al., Southeast Asian J Trop. Med. Public Health, 24(4): 624-30 (1993). Iron administration was studied, among others, in calves, e.g. see: Geisser, P. et al., Arzneimittelforschung, 41(1): 32-37 (1991).

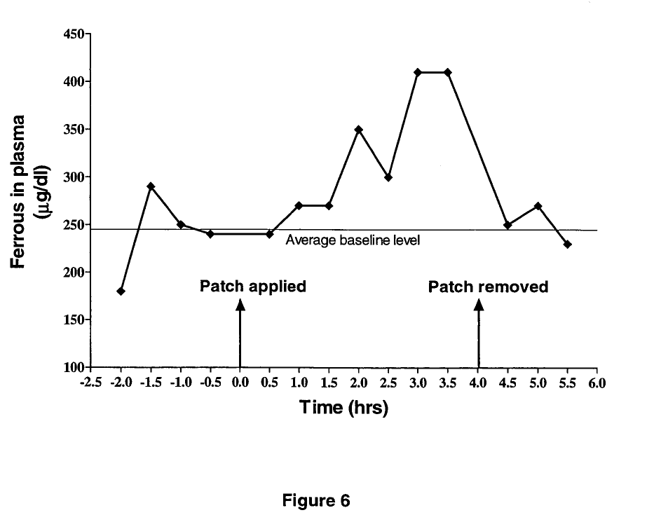

A patch of the novel PTF was soaked with an emulsion of the present invention comprising of a non-oily emulsion, MSM and ferrous sulphate (salt concentration should be adjusted to the range 10-20%). The patch was applied to the ears of three calves whose average ferrous concentration in plasma was 245 μg/dl. After 3.5 hours ferrous plasma concentration reached a level of 410 μg/dl (see FIG. 6). Ferrous plasma levels declined rapidly after removal of the patch (4.6 hours following application).

### EXAMPLE 8

The PTF is obviously capable also of inducing the penetration of small molecules and drugs through the skin. The NSAID ibuprofen, was studied, as many other drugs, for its percutaneous bioavailability, e.g. see: Kleinbloesem, C. H., et a., Arzneimittelforschung, 45(10): 1117-21 (1995).

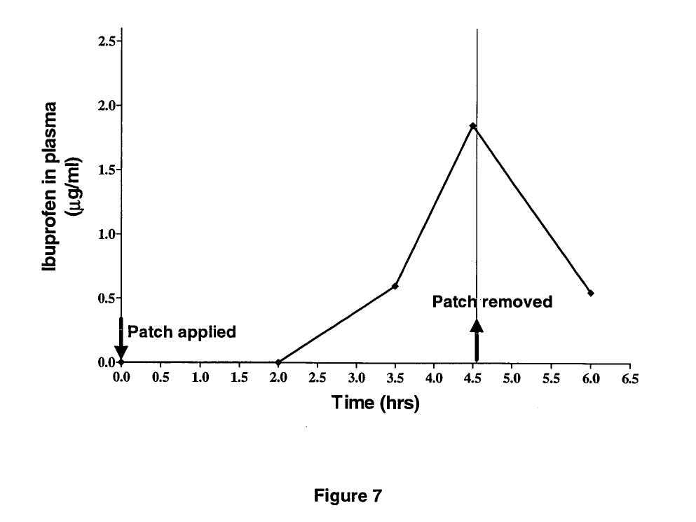

A pad of the novel PTF with the non-oily emulsion containing ibuprofen chloride was applied to the skin of three rabbits (see FIG. 7).

Adjustment of plasma levels to a preferred therapeutic concentration (around 10 μg/ml) can be easily achieved by varying the concentration of ibuprofen in the mixture and/or the size of the patch, as is the common practice in transdermal patches.

### EXAMPLE 9

Human calcitonin is a 32 amino acid peptide hormone (MW 3,527), synthesized in the C-cells of the thyroid gland. Calcitonins (especially salmon calcitonin, MW 3,432) have been recognized as effective drugs for several diseases, including hypercalcemia, Paget's disease and osteoporosis. Calcitonins are rapidly inactivated when given by mouth and therefore their administration relies on either parenteral injection or recently also nasal spray. Intensive efforts have been devoted to the transdermal (mainly iontophoretic) delivery of salmon calcitonin, e.g. see: Chang, S L et a., Intern. J. Pharmac. 200:107-113 (2000).

During the last few years, the importance of daily low-dose intermittent treatments with parathyroid hormone (hPTH 1-34) for the increase in bone formation in postmenopausal women has immerged, e.g. see: Rehman, Q et al., Osteoporos Int, 14:77-81(2003). To avoid the inconvenient daily injections of this 34 amino acid peptide hormone, which markedly affected compliance, attempts had also been made to deliver it transdermally, e.g. through the use of pulsatile iontophoresis, see: Suzuki, Yet al., J. Pharm. Pharmacol. 53:1227-1234 (2001). The possibility of sequential therapy with the two hormones, PTH and calcitonin, was also suggested.

The novel PTF provides a new approach to enable a simple and convenient transdermal delivery of these closely associated hormones, for the treatment and prevention of osteoporosis. To prove the feasibility of the proposed method, a study of the transdermal delivery of calcitonin was initiated in calves.

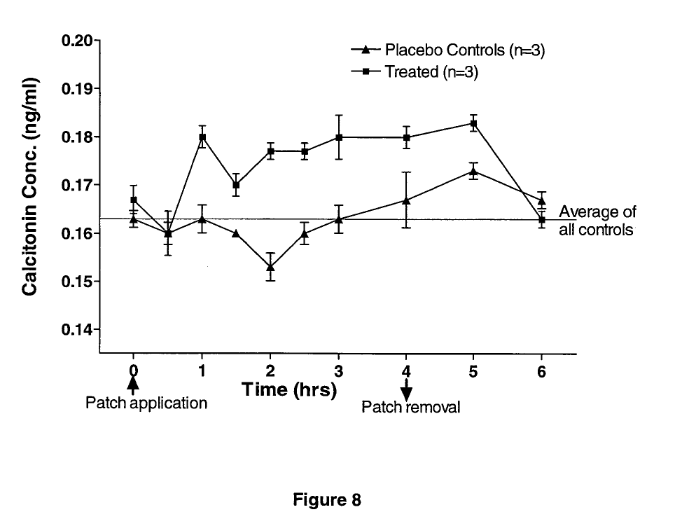

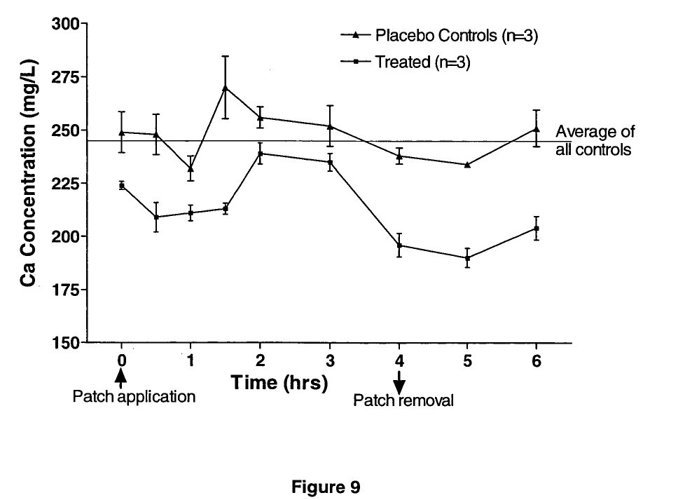

A patch containing the novel PTF with 600 IU of salmon calcitonin and a protease inhibitor was applied to the ears of three calves whose average calcitonin-like immunoreactivity in plasma was 163 pg/ml. Three other calves were treated with control placebo patches. The patches were applied for 4 hours. One hour after calcitonin patch application and up to one hour following removal of the patch, calcitonin immunoreactivity in the plasma of the treated calves was higher than that found in the placebo treated (see FIG. 8). In addition, the physiological effect of calcitonins, namely lowering of calcium concentration in plasma, was also recorded (see FIG. 9), especially during two hours following the 4 hours patch treatment.

### EXAMPLE 10

Parasitic infections, caused by pathogenic protozoa or helminths (worms—nematodes, trematodes or cestodes), affect over 3 billion people all around the world, with helminthiasis itself affecting over 2 billion, particularly in tropical regions. Due to the intensive human travel and migration of our age, there is a realistic threat of worms spreading to geographic locations that previously had been considered free of the parasites. Parasites infect also domestic animals to a large extent (e.g. flukes), imposing a substantial health and economic burden.

Many antiparasitic agents were developed originally for veterinary use and only later were adapted to human beings. One example is ivermectin (MW 875), an insoluble drug used extensively to control and treat a broad spectrum of infections caused by parasitic nematodes and arthropods (insects, ticks, and mites) that infect livestock and domestic animals. It was also recently found to be quite successful in the human treatment of scabies. Triclabendazole (TCBZ), another insoluble drug used successfully in veterinary medicine, showed considerable promise for treating human infections (e.g. paragonimiasis, enterobiasis etc.). Formulations of ivermectin with triclabendazole were shown to be very effective against liver fluke (Fasciola hepatica), gastrointestinal nematodes in cattle and sheep and sucking lice species in cattle. Another common parasiticide, emetine, is a drug used to treat infections of the liver, bowel and intestine caused by amoebae, including ameobic dysentery. It is a bitter and somewhat poisonous alkaloid that is administered by injection (which may be painful) and irritates the stomach lining and other mucous membranes.

The transdermal application of parasticides can offer an excellent solution to many drug administration difficulties and have an enormously important economic value for the use in livestock and domestic animals.

Another important use of the transdermal application is for specific cases of antibiotic treatment. In several gastrointestinal pathological states (e.g. the use of erythromycin for the treatment of gastroparesis) the transdermal route may offer an optimal solution to otherwise erratic drug administration.

To demonstrate the value of the novel PTF for treatment with antiparasitic agents, a solution of 400 mg/ml of TCBZ in the PTF was used. The study was carried out in 5 cows, around 200 kg each, treated transdermally with 6 ml. Blood samples were taken during the 5 day study, and the patch was removed about 18 hours before the last sample. TCBZ was determined by extraction of plasma samples and reversed phase HPLC, using UV-detection.

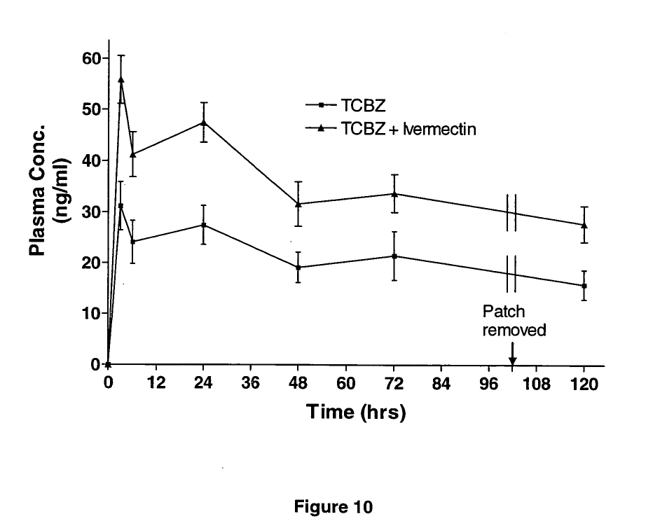

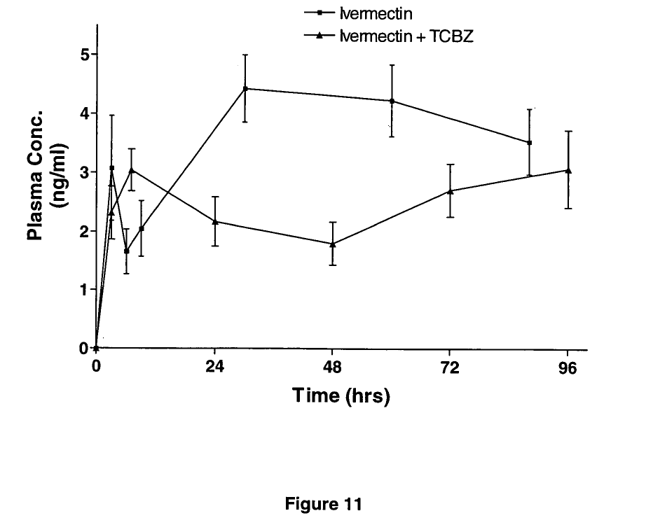

TCBZ is metabolized rapidly to its sulphoxide (TCBZ-SO) and sulphone (TCBZ-SO2) derivatives following administration, active metabolites that are eliminated slowly. Following oral dosing, very little, if any, unchanged drug is detected in animals plasma. In the current study, following transdermal application (see FIG. 10), considerable amount of TCBZ was detected in the first sample, 3 hours after application of the patch. The concentration of the drug was quite constant for 72 hours, somewhat dropping only in the last sample, 18 hours after patch removal.

To test the possibility of a combined TCBZ+ivermectin treatment, 5 similar cows were treated with PTF patch containing both TCBZ and 100 mg/ml of ivermectin. The pharmacokinetic profile was quite similar (see FIG. 10), only this time TCBZ concentrations were about 70% higher. In additional 5 cows, ivermectin alone was used and compared to the mixture ivermectin+TCBZ (see FIG. 11). Though ivermectine is persistently insoluble and it has quite a large molecular weight, plasma analysis exhibited continuously stable concentrations of the drug for the duration of the 4 days study. In the drug combination of ivermectin TCBZ, the concentration of ivermectin was somewhat lower on the second day of treatment, but otherwise reasonably stable over the treatment period.

The non-oily emulsion of the present invention provides a platform for transdermal formulations that are universally applicable and allows the manufacture of medicaments for transdermal administration of small molecules, ionic compounds, antiparasitic agents, anthelmintics, antibiotics and/or polypeptides with a molecular weight of up to 7000 Dalton, for human treatment and/or for the treatment of animals.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

1. A composition for transdermal administration of at least one therapeutically active compound or nutrient, said composition comprising:
at least one item selected from the group consisting of at least one therapeutically active compound and at least one nutrient; and
a non-oily emulsion.

2. The composition for transdermal administration according to claim 1, wherein said at least one therapeutically active compound and said at least one nutrient is an ionic compound.

3. The composition for transdermal administration according to claim 2, wherein the ionic compound is a metal ion.

4. The composition according to claim 1, wherein said at least one therapeutically active compound is a polypeptide.

5. The composition according to claim 4, wherein said polypeptide has a molecular weight of up to 7000 kDa.

6. The composition according to claim 1, wherein said at least one therapeutically active compound is selected from the group consisting of antiparasitic agents, anthelmintic drugs and antibiotic drugs, used for the treatment of humans, livestock or domestic animals.

7. The composition according to any one claim 1, wherein said non-oily emulsion is a mixture of lecithin, bile salt and cholesterol.

8. The composition according to claim 7, wherein, said lecithin is present in said non-oily emulsion in an amount of 2-15% (w/v), said bile salt is present in said non-oily emulsion in an amount of 2-15% (w/v), and said cholesterol is present in said non-oily emulsion in an amount of 2-15% (w/v).

9. The composition according to claim 7, wherein the ratio by weight of lecithins, bile salts and cholesterol is 2:1:1.

10. The composition according to claim 8, wherein the sum of the amounts of lecithins, bile salts and cholesterol constitutes 6-30% (w/v) of the non-oily emulsion.

11. The composition according to claim 1, wherein the composition further comprises an organic sulfur compound.

12. The composition according to claim 11, wherein, the organic sulfur compound is present in said composition in an amount of 2-30% (w/v), in relation to the non-oily emulsion.

13. The composition according to claim 11, wherein the organic sulfur compound is selected from the group consisting of dimethylsulfoxide, methylsulfonylmethane, 2,3-dimethylsulfolane, 2,4-dimethylsulfolane and sodium lauryl sulfate.

14. Use of the composition according to claim 1 for the manufacture of a cream, gel, lotion, suppositories, ointment, patch (TTS) for transdermal administration of active substances, preferably nutrients and/or medications.

15. Use of the composition according to claim 1 for transdermal administration of active substances, preferably nutrients and/or medications.

16. The composition according to claim 12, wherein the organic sulphur compound is present in said composition in an amount of 4-25% (w/v), in relation to said non-oily emulsion.
